(19) United States
(12) Patent Application Publication
(10) Pub. No.: US 2009/0105764 A1
(43) Pub. Date: Apr. 23, 2009

(54) Dynamic stabilization member with fin support and solid core extension

(76) Inventor: Roger P. Jackson, Prairie Village, KS (US)

Correspondence Address: LAW OFFICE OF JOHN C. MCMAHON, P.O. BOX 30069, KANSAS CITY, MO, 64112, US

(21) Appl. No.: 12/288,479

(22) Filed: Oct. 21, 2008

Related U.S. Application Data
(60) Provisional application No. 61/000,232, filed on Oct. 24, 2007.
(60) Provisional application No. 60/999,965, filed on Oct. 23, 2007.

(51) Int. Cl. A61B 17/70; A61B 17/04

(52) U.S. Cl. 606/264; 606/246; 623/17.11; 606/301

(57) ABSTRACT

A dynamic fixation medical implant having at least two bone anchors includes a dynamic longitudinal connecting member assembly having the following features: a pair of elongate segments, each segment having at least one and up to a plurality of integral fins axially extending therefrom; a core extension integral with one of the elongate segments and slidingly received in the other elongate segment; a molded spacer that substantially surrounds the fins and may partially or substantially surround the abutment plates; an optional bumper; an optional crimp ring; and optional sleeves having abutment plates and fins for placement between elongate segments.

### CROSS-REFERENCE TO RELATED APPLICATIONS

This application claims the benefit of U.S. Provisional Patent Application Ser. No. 61/000,232, filed Oct. 24, 2007 and also the benefit of U.S. Provisional Patent Application Ser. No. 60/999,965, filed Oct. 23, 2007, both of which are incorporated by reference herein.

### BACKGROUND OF THE INVENTION

The present invention is directed to dynamic fixation assemblies for use in bone surgery, particularly spinal surgery, and in particular to stiff, telescoping longitudinal connecting members and cooperating bone anchors or fasteners for such assemblies, the connecting members being attached to at least two bone anchors.

Historically, it has been common to fuse adjacent vertebrae that are placed in fixed relation by the installation therealong of bone screws or other bone anchors and cooperating longitudinal connecting members or other elongate members. Fusion results in the permanent immobilization of one or more of the intervertebral joints. Because the anchoring of bone screws, hooks and other types of anchors directly to a vertebra can result in significant forces being placed on the vertebra, and such forces may ultimately result in the loosening of the bone screw or other anchor from the vertebra, fusion allows for the growth and development of a bone counterpart to the longitudinal connecting member that can maintain the spine in the desired position even if the implants ultimately fail or are removed. Because fusion has been a desired component of spinal stabilization procedures, longitudinal connecting members have been designed that are of a material, size and shape to largely resist flexure, extension, torsion, distraction and compression, and thus substantially immobilize the portion of the spine that is to be fused. Thus, longitudinal connecting members are typically uniform along an entire length thereof, and usually made from a single or integral piece of material having a uniform diameter or width of a size to provide substantially rigid support in all planes.

Fusion, however, has some undesirable side effects. One apparent side effect is the immobilization of a portion of the spine. Furthermore, although fusion may result in a strengthened portion of the spine, it also has been linked to more rapid degeneration and even hyper-mobility and collapse of spinal motion segments that are adjacent to the portion of the spine being fused, reducing or eliminating the ability of such spinal joints to move in a more normal relation to one another. In certain instances, fusion has also failed to provide pain relief.

An alternative to fusion and the use of more rigid longitudinal connecting members or other rigid structure has been a “soft” or “dynamic” stabilization approach in which a flexible loop-, S-, C- or U-shaped member or a coil-like and/or a spring-like member is utilized as an elastic longitudinal connecting member fixed between a pair of pedicle screws in an attempt to create, as much as possible, a normal loading pattern between the vertebrae in flexion, extension, distraction, compression, side bending and torsion. Problems may arise with such devices, however, including tissue scarring, lack of adequate spinal support or being undesirably large or bulky when sized to provide adequate support, and lack of fatigue strength or endurance limit. Fatigue strength has been defined as the repeated loading and unloading of a specific stress on a material structure until it fails. Fatigue strength can be tensile or distraction, compression, shear, torsion, bending, or a combination of these.

Another type of soft or dynamic system known in the art includes bone anchors connected by flexible cords, straps or strands, typically made from a plastic material. Such a cord, strap or strand may be threaded through cannulated compressible spacers that are disposed between adjacent bone anchors when such a cord or strand is implanted, tensioned and attached to the bone anchors. The spacers typically span the distance between bone anchors, providing limits on the bending movement of the cord or strand and thus strengthening and supporting the overall system. Such cord or strand-type systems require specialized bone anchors and tooling for tensioning and holding the chord or strand in the bone anchors. Although flexible and compressible, the cords or strands utilized in such systems do not allow for elastic distraction or any elongation of the system once implanted because the cord or strand must be stretched or pulled to maximum tension in order to provide a stable, supportive system. Also, as currently designed, these systems do not provide any significant torsional and/or shear resistance.

The complex dynamic conditions associated with spinal movement therefore provide quite a challenge for the design of elongate longitudinal connecting members that exhibit an adequate fatigue strength to provide stabilization and protected motion of the spine, without fusion, and allow for some natural movement of the portion of the spine being reinforced and supported by the elongate connecting member. A further challenge are situations in which a portion or length of the spine requires a more rigid or stiff stabilization, possibly including fusion, while another portion or length may be better supported by a more dynamic system that allows for protective cephalad and caudad movement or translation along a solid stiff longitudinal connecting member which also resists shear stresses.

### SUMMARY OF THE INVENTION

Longitudinal connecting member assemblies according to the invention for use between at least two bone anchors provide dynamic, protected motion of the spine and may be extended to provide additional dynamic sections or more stiff support along an adjacent length of the spine, with fusion, if desired. A longitudinal connecting member assembly according to the invention includes first and second stiff elongate segments, each segment having an abutment plate with a plurality of integral fins extending axially from the abutment plate. The fins face one-another and are evenly spaced from one another and are also evenly spaced from the opposing plate. The first connecting member body further includes an elongate central inner solid stiff core extension that extends axially between the fins and also through the second connecting member. The first connecting member stiff core extension can have a decreased cross-sectional area along a length thereof to cooperate in a sliding relationship with the stiff second connecting member. The first connecting member fins may be integral with the core extension. The assembly further includes an elastic molded outer spacer or elastomer sleeve disposed about the fins and may further completely surround each of the plates. The fins may be cupped or hooked to further grab and hold the elastomer. The assembly may further include an optional elastic end bumper that can place and maintain a distractive force on the elongate stiff and non-stretchable solid inner core. The cupped fins and/or over-molded elastomer around the abutment plates prevent or eliminate gapping or pulling away of the plate from the elastic polymer so that soft tissues and body fluids can not get into this space with axial translations along the implant.

### OBJECTS AND ADVANTAGES OF THE INVENTION

An object of the invention is to provide dynamic medical implant stabilization assemblies having stiff longitudinal connecting members that resist shear forces and yet allow torsion, compression and distraction displacements of the assembly. A further object of the invention is to provide dynamic medical implant longitudinal connecting members that may be utilized with a variety of bone screws, hooks and other bone anchors. Another object of the invention is to provide a solid stiffer connecting member portion or segment, if desired, with a different cross-sectional area integral with the solid stiff core extension portion. Additionally, it is an object of the invention to provide a lightweight, reduced volume, low profile assembly including at least two bone anchors and a longitudinal connecting member assembly therebetween. Furthermore, it is an object of the invention to provide apparatus and methods that are easy to use and especially adapted for the intended use thereof and wherein the apparatus are comparatively inexpensive to make and suitable for use.

Other objects and advantages of this invention will become apparent from the following description taken in conjunction with the accompanying drawings wherein are set forth, by way of illustration and example, certain embodiments of this invention.

The drawings constitute a part of this specification and include exemplary embodiments of the present invention and illustrate various objects and features thereof.

### BRIEF DESCRIPTION OF THE DRAWINGS

FIG. 1 is an enlarged and exploded front elevational view of a dynamic fixation connecting member assembly according to the invention including first and second elongate members, each with a finned plate, an elongate core member integral with the first member, an elastic bumper, a crimping ring and an outer molded spacer (not shown).

FIG. 2 is an enlarged perspective view of the assembly of FIG. 1 without the bumper, crimping ring and molded spacer.

FIG. 3 is an enlarged front elevational view of the assembly of FIG. 1, shown assembled.

FIG. 4 is an enlarged front elevational view, similar to FIG. 3, with portions broken away to show the detail thereof and the molded spacer shown in phantom.

FIG. 5 is an enlarged front elevational view of the assembly of FIG. 1, shown assembled and with the molded spacer.

FIG. 6 is a reduced front elevational view of the assembly of FIG. 5 shown with three bone screws.

FIG. 7 is an enlarged front elevational view of an alternative embodiment of a dynamic fixation connecting member assembly according to the invention including first and second finned elongate members, an elongate core member integral with the first member, an elastic bumper, a crimping ring a finned sleeve or tube trolley and two outer molded spacers.

FIG. 8 is an enlarged front elevational view of the assembly of FIG. 7 with portions broken away to show the detail thereof.

FIG. 9 is an enlarged front elevational view of the sleeve or tube trolley of FIG. 7.

FIG. 10 is a reduced front elevational view of the assembly of FIG. 7 shown with three bone screws.

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure. It is also noted that any reference to the words top, bottom, up and down, and the like, in this application refers to the alignment shown in the various drawings, as well as the normal connotations applied to such devices, and is not intended to restrict positioning of the connecting member assemblies of the application and cooperating bone anchors in actual use.

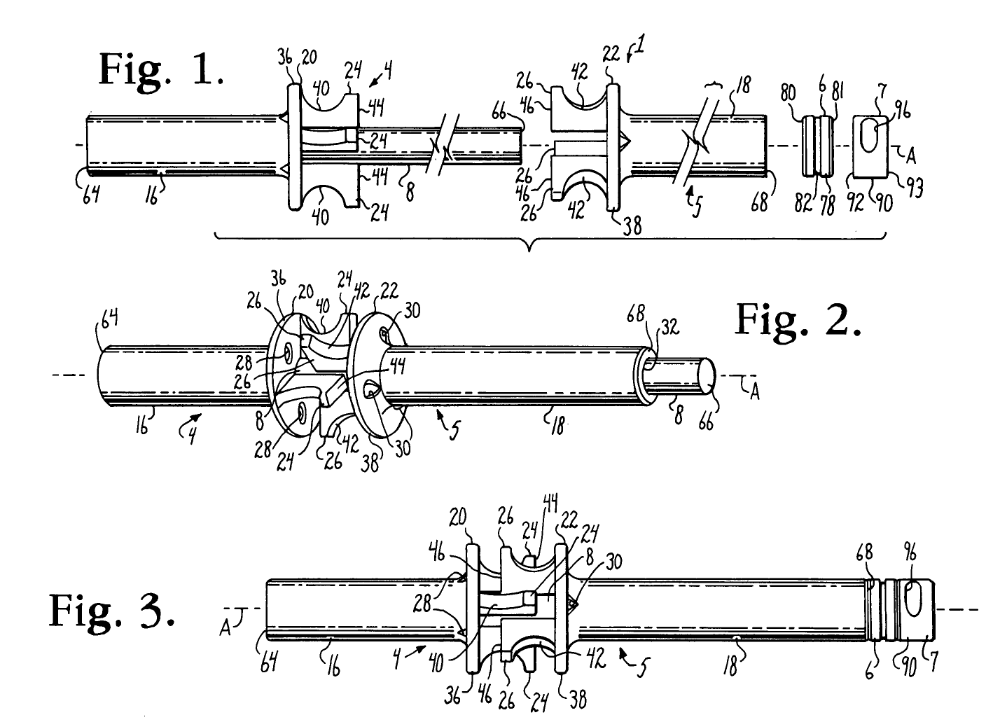

With reference to FIGS. 1-6, the reference numeral 1 generally designates a non-fusion dynamic stabilization longitudinal connecting member assembly according to the present invention. The connecting member assembly 1 includes first and second elongate segments, generally 4 and 5, an elastic bumper 6 and a crimping ring 7. The elongate segment 4 further includes a solid stiff inner core extension 8. The assembly further includes an outer sleeve or spacer 10. The illustrated core 8 is cylindrical and substantially solid, having a central longitudinal axis A that is also the central longitudinal axis A of the entire assembly 1 when the spacer 10 is molded thereon, connecting the segments 4 and 5. The core 8 provides stability to the assembly 1, particularly with respect to torsional and shear stresses placed thereon. The solid core 8 may be tensioned prior to molding of the spacer 10; however, it is stiff and does not stretch.

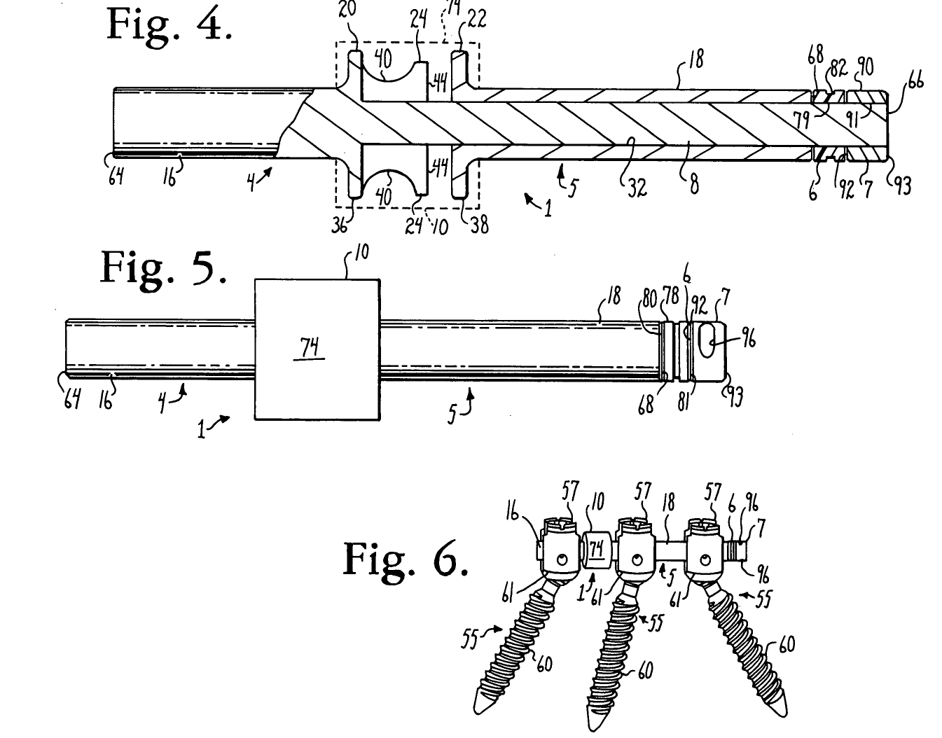

With particular reference to FIGS. 1-4 the elongate segments 4 and 5 further include respective bone attachment end portions 16 and 18, respective end plates 20 and 22 having respective integral hooked fin or wing members 24 and 26. In the illustrated embodiment, there are three equally spaced fins 24 and 26 extending generally along the axis A from the respective plates 20 and 22. However, in other embodiments according to the invention there may be more than three or less than three hooked fins 24 and 26. Each plate 20 and 22 also includes three apertures or through bores 28 and 30, respectively, spaced substantially equally between the respective fins 24 and 26. The through bores 28 and 30 extend substantially parallel to the axis A. The central core 8 is integral with the plate 20 and extends along the central axis A and between both sets of fins 24 and 26. The core 8 may also be integral with the fins 24. As best shown in FIGS. 2 and 4, the core 8 also extends through an axial through bore 32 of the segment 5.

As best shown in FIGS. 1-3, each of the hooked fins 24, as well as the hooked fins 26, extend axially away from the respective plate 20, 22 (along the axis A) and also extend radially from near the core 8 to or substantially near a respective outer peripheral substantially cylindrical surface 36 and 38 of the respective plates 20 and 22. Near the peripheral surfaces 36 and 38, the respective fins 24 and 26 include a curved concave or C-shaped hooked surface 40 and 42, respectively, such surface facing outwardly away from the axis A and running from the respective plates 20 and 22 to near respective end surfaces 44 and 46. When the segments 4 and 5 are assembled and set in place by the molded spacer 10, the surfaces 44 are near and in substantially uniform spaced relation with the plate 22 and the surfaces 46 are near and in substantially uniform spaced relation with the plate 20. The hooked surfaces 40 and 42 provide structure for mechanical cooperation and attachment with the molded spacer 10 as will be discussed in greater detail below. Also, as will be described in greater detail below, the spacer 10 is molded about the hooked fins 24 and 26, about the core 8 located between the plates 20 and 22, and through the apertures or bores 28 and 30 of the respective plates 20 and 22 in a manner so as to result in a mechanically connected structure, the elastomeric material completely surrounding the plates 20 and 22 as well as the fins 24 and 26. In certain embodiments, the elastomeric material of the molded spacer 10 may also adhere to fin, core extension and plate surfaces. An adhesive may also be added to provide such adherence between the spacer 10 and the plates and fins. Alternatively, in certain embodiments a coating or sleeve may be placed around the core 8 portion located between the plates 20 and 22 prior to molding so that the core 8 is spaced from the spacer 10 and thus slidably movable with respect to the spacer 10.

The dynamic connecting member assembly 1 cooperates with at least a pair of bone anchors (three shown in FIG. 6), such as the polyaxial bone screws, generally 55 and cooperating closure structures 57 shown in FIG. 6, the assembly 1 being captured and fixed in place at the end portions 16 and 18 by cooperation between the bone screws 55 and the closure structures 57 with the spacer 10 being disposed between an adjacent pair of the bone screws 55.

Because the illustrated end portions 16 and 18 are stiff and cylindrical, the connecting member assembly 1 may be used with a wide variety of bone anchors already available for cooperation with rigid rods including fixed, monoaxial bone screws, hinged bone screws, polyaxial bone screws, and bone hooks and the like, with or without one or more compression inserts, that may in turn cooperate with a variety of closure structures having threads, flanges, or other structure for fixing the closure structure to the bone anchor, and may include other features, for example, break-off tops and inner set screws, as well as associated pressure inserts. It is foreseen that the portions 16 and 18 may in other embodiments of the invention have larger and smaller diameters and other cross-sectional shapes, including, but not limited to oval, square, rectangular and other curved or polygonal shapes. The bone anchors, closure structures and the connecting member assembly 1 are then operably incorporated in an overall spinal implant system for correcting degenerative conditions, deformities, injuries, or defects to the spinal column of a patient.

The illustrated polyaxial bone screws 55 each include a shank 60 for insertion into a vertebra (not shown), the shank 60 being pivotally attached to an open receiver or head 61. The shank 60 includes a threaded outer surface and may further include a central cannula or through-bore disposed along an axis of rotation of the shank to provide a passage through the shank interior for a length of wire or pin inserted into the vertebra prior to the insertion of the shank 60, the wire or pin providing a guide for insertion of the shank 60 into the vertebra. The receiver 61 has a pair of spaced and generally parallel arms that form an open generally U-shaped channel therebetween that is open at distal ends of the arms. The arms each include radially inward or interior surfaces that have a discontinuous guide and advancement structure mateable with cooperating structure on the closure structure 57. The guide and advancement structure may take a variety of forms including a partial helically wound flangeform, a buttress thread, a square thread, a reverse angle thread or other thread like or non-thread like helically wound advancement structure for operably guiding under rotation and advancing the closure structure 57 downward between the receiver 61 arms and having such a nature as to resist splaying of the arms when the closure 57 is advanced into the U-shaped channel. For example, a flange form on the illustrated closure 57 and cooperating structure on the arms of the receiver 61 is disclosed in Applicant's U.S. Pat. No. 6,726,689, which is incorporated herein by reference.

The shank 60 and the receiver 61 may be attached in a variety of ways. For example, a spline capture connection as described in U.S. Pat. No. 6,716,214, and incorporated by reference herein, is used for the embodiment disclosed herein. Polyaxial bone screws with other types of capture connections may also be used according to the invention, including but not limited to, threaded connections, frictional connections utilizing frusto-conical or polyhedral capture structures, integral top or downloadable shanks, and the like. Also, as indicated above, polyaxial and other bone screws for use with connecting members of the invention may have bone screw shanks that attach directly to the segments 16 and 18 may include compression members or inserts that cooperate with the bone screw shank, receiver and closure structure to secure the connecting member assembly to the bone screw and/or fix the bone screw shank at a desired angle with respect to the bone screw receiver that holds the longitudinal connecting member assembly. Furthermore, although the closure structure 57 of the present invention is illustrated with the polyaxial bone screw 55 having an open receiver or head 61, it foreseen that a variety of closure structure may be used in conjunction with any type of medical implant having an open or closed head, including monoaxial bone screws, hinged bone screws, hooks and the like used in spinal surgery.

To provide a biologically active interface with the bone, the threaded shank 60 may be coated, perforated, made porous or otherwise treated. The treatment may include, but is not limited to a plasma spray coating or other type of coating of a metal or, for example, a calcium phosphate; or a roughening, perforation or indentation in the shank surface, such as by sputtering, sand blasting or acid etching, that allows for bony ingrowth or ongrowth. Certain metal coatings act as a scaffold for bone ingrowth. Bio-ceramic calcium phosphate coatings include, but are not limited to: alpha-tri-calcium phosphate and beta-tri-calcium phosphate (Ca₃ (PO₄)₂, tetra-calcium phosphate (Ca₄P₂O₉), amorphous calcium phosphate and hydroxyapatite (Ca₁₀(PO₄)₆(OH)₂). Coating with hydroxyapatite, for example, is desirable as hydroxyapatite is chemically similar to bone with respect to mineral content and has been identified as being bioactive and thus not only supportive of bone ingrowth, but actively taking part in bone bonding.

The closure structure 57 can be any of a variety of different types of closure structures for use in conjunction with the present invention with suitable mating structure on the interior surface of the upstanding arms the receiver 61. The illustrated closure structure 57 is rotatable between the spaced receiver arms, but could be a twist-in or a slide-in closure structure. The closure 57 includes an outer helically wound guide and advancement structure in the form of a flange form that operably joins with the guide and advancement structure disposed on the interior of the arms of the receiver 61. The illustrated closure structure 57 includes a lower or bottom surface that is substantially planar and may include a point and/or a rim protruding therefrom for engaging the portion 16 or 18 outer cylindrical surface. The closure structure 57 has a top surface with an internal drive feature, that may be, for example, a star-shaped drive aperture sold under the trademark TORX. A driving tool (not shown) sized and shaped for engagement with the internal drive feature is used for both rotatable engagement and, if needed, disengagement of the closure 57 from the arms of the receiver 61. The tool engagement structure may take a variety of forms and may include, but is not limited to, a hex shape or other features or apertures, such as slotted, tri-wing, spanner, two or more apertures of various shapes, and the like. It is also foreseen that the closure structure 57 may alternatively include a break-off head designed to allow such a head to break from a base of the closure at a preselected torque, for example, 70 to 140 inch pounds. Such a closure structure would also include a base having an internal drive to be used for closure removal.

The longitudinal connecting member assembly 1 illustrated in FIGS. 1-6 is elongate, with the attachment portion 16, the plate 20, the core 8 and the fins 24 being integral and the attachment portion 18, the plate 22 and the fins 26 being integral. The inner core 8 is slidingly received in the portion 18. The stiff segments 4 and 5 and the solid stiff core 8 are preferably made from metal, metal alloys, such as cobalt chrome, or other suitable materials, including stiff plastic polymers such as polyetheretherketone (PEEK), ultra-high-molecular weight-polyethylene (UHMWP), polyurethanes and composites. The elastomeric molded spacer 10 may be made of a variety of materials including plastics and composites. The illustrated spacer 10 is a molded thermoplastic elastomer, for example, polyurethane or a polyurethane blend; however, any suitable polymer material may be used.

Specifically, in the illustrated embodiment, the core 8 and the end portion 16 are substantially solid, stiff and smooth uniform cylinders or rods, each of a uniform circular cross-section, which, in the embodiment shown, have different diameters. The end portion 18 is tubular with inner and outer circular cross-sections, and also having an outer profile that is a smooth uniform cylinder having an outer diameter, which in the embodiment shown, is the same as the outer diameter of the portion 16. The tubular end portion 18 terminates at an end 68. The portions 16 and 18 are each sized and shaped to be received in the channel formed between arms of a bone screw receiver 61 with the plates 20 and 22 and the molded spacer 10 disposed between cooperating adjacent bone screws 55. Prior to final assembly, the core 8 is typically of a length greater than that shown in the drawing figures so that the core 8 may be grasped by a tool (not shown) near the end 66 and pulled along the axis A in a direction away from the attachment portion 16 in order to place tension on the core 8.

The spacer 10 advantageously cooperates with the plates 20 and 22, the fins 24 and 26 and the core 8 to provide an element or segment that allows for torsion, compression and distraction of the assembly 1. The spacer 10 further provides a smooth substantially cylindrical surface that protects a patient's body tissue from damage that might otherwise occur with, for example, a spring-like dynamic member. The over-molded elastomer also prevents soft tissues, including scar tissue, from getting between the plates and polymer.

The molded spacer 10 is fabricated about the plates 20 and 22 and the fins 24 and 26, as will be described more fully below, and in the presence of the core 8, with molded plastic flowing about the plates and fins. The formed elastomer is substantially cylindrical in outer form with an external substantially cylindrical surface 74 that has the same or substantially similar diameter as the diameter of the outer cylindrical surfaces 36 and 38 of the respective stop or abutment plates 20 and 22. It is foreseen that in some embodiments, the spacer may be molded to be of square, rectangular or other outer and inner cross-sections including curved or polygonal shapes. The portion 16, portion 18 and inner solid core 8 may also be of other cross-sections including, but not limited to, square, rectangular and other outer and inner cross-sections, including curved or polygonal shapes. The spacer 10 may further include one or more compression grooves (not shown) formed in the surface 74. During the molding process a sleeve or other material (not shown) may be placed about the core 8 so that the spacer 10 has in internal surface of a slightly greater diameter than an outer diameter of the core 8, allowing for axially directed sliding movement of the spacer 10 with respect to the core 8.

With reference to FIGS. 1,3, 4 and 5, the bumper 6 is substantially cylindrical, including an outer surface 78 and an inner surface 79 forming a substantially cylindrical through bore that opens at planar opposed end surfaces 80 and 81 and operatively extends along the axis A. The bumper 6 further includes an optional compression groove 82. The bumper 6 is sized and shaped to slidingly receive the core 8 through the inner surface 79. The bumper 6 is preferably made from an elastomeric material such as polyurethane. The bumper 6 operatively provides axial tension on the core 8 as will be described in greater detail below.

Also with particular reference to FIGS. 1, 3, 4 and 5, the crimping ring 7 is substantially cylindrical and includes an outer surface 90 and an inner surface 91 forming a substantially cylindrical through bore that opens at opposed planar end surfaces 92 and 93 and operatively extends along the axis A. The crimping ring 7 is sized and shaped to receive the elongate core 9 through the inner surface 91. The crimping ring 7 further includes a pair of crimp or compression grooves 96 that are pressable and deformable inwardly toward the axis A upon final tensioning of the core 8 and the spacer 10 during assembly of the assembly 1. The crimping ring 7 is preferably made from a stiff, but deformable material, including metals and metal alloys.

In use, at least two bone screws 55 are implanted into vertebrae for use with the longitudinal connecting member assembly 1. Each vertebra may be pre-drilled to minimize stressing the bone. Furthermore, when a cannulated bone screw shank is utilized, each vertebra will have a guide wire or pin (not shown) inserted therein that is shaped for the bone screw cannula of the bone screw shank 60 and provides a guide for the placement and angle of the shank 60 with respect to the cooperating vertebra. A further tap hole may be made and the shank 60 is then driven into the vertebra by rotation of a driving tool (not shown) that engages a driving feature at or near a top of the shank 60. It is foreseen that the screws 55 and the longitudinal connecting member 1 can be inserted in a percutaneous or minimally invasive surgical manner.

The longitudinal connecting member assembly 1 may be assembled to provide a pre-tensioned core 8 and pre-compressed spacer 10 and bumper 6 prior to implanting the assembly 1 in a patient. This is accomplished by first providing the segment 4 that has the core 8 that is longer in the axial direction A than the core 8 illustrated in the drawing figures. The segment 5 is then threaded onto the core 8 with the fins 26 of the plate 22 facing the fins 24 of the segment 4. The core 8 is received in the bore 32 and the segment 5 is moved along the core 8 toward the plate 20. The fins 24 and 26 are manipulated to be evenly spaced from one another with a desired uniform substantially equal space between the fin ends 46 and the plate 20 and the fin ends 44 and the plate 22. This is performed in a factory setting with the end portions 16 and 18 held in a jig or other holding mechanism that frictionally engages and holds the sections 16 and 18, for example, and the spacer 10 is molded about the plates 20 and 22 as well as the fins 24 and 26 as shown in phantom in FIG. 4. The elastomer of the spacer 10 flows through the plate through bores 28 and 30 as well as around and about each of the fins 24 and 26, the resulting molded spacer 10 surrounding all of the surfaces of the plates 20 and 22 as well as all of the surfaces of the fins 24 and 26. If desired, prior to molding, a sheath or coating may be placed about the core 8 so that the spacer 10 material does not contact the core 8. However, in other embodiments of the invention, the elastomer is allowed to flow about and contact the core 8, that may be pre-tensioned or tensioned after the molding process. The jig or holding mechanism may then be released from the portions 16 and 18 after the molding of the spacer 10 is completed. The portions 16 and 18 may be held in a straight or angled position.

Either before or after molding, the bumper 6 is loaded onto the core 8 by inserting the core 8 end 66 into the bore defined by the inner surface 79 with the face 80 facing the toward the surface 68 of the portion 18. The bumper 6 is moved along the core 8 until the surface 80 contacts the surface 68. The crimping ring 7 is thereafter loaded onto the core 8 by inserting the core 8 end 66 into the bore defined by the inner surface 91 with the face 92 facing the toward the surface 81 of the bumper 6. The crimping ring 7 is moved along the core 8 until the surface 92 contacts the surface 81. It is noted that due to the symmetrical nature of the bumper 6 and the crimping ring 7, these components may be loaded onto the core 8 from either side thereof.

After the crimping ring 7 is loaded onto the core 8, manipulation tools (not shown) are used to grasp the core 8 near the end 66 and at the bone anchor attachment portion 16, placing tension on the core 8. Furthermore, the spacer 10 and/or the bumper 6 are compressed, followed by deforming the crimping ring, or otherwise fixing an end stop on the core, at the crimp grooves 96 and against the core 8. When the manipulation tools are released, the crimping ring 7, or fixed end stop now firmly and fixedly attached to the core 8 holds the spacer 10 and/or the bumper 6 in compression and the spacer and/or the bumper places axial tension forces on the core 8, resulting in an axial dynamic relationship between the core 8 and the spacer 10 and/or the bumper 6.

With reference to FIG. 6, the assembly 1 is eventually positioned in an open or percutaneous manner in cooperation with the at least two bone screws 55 and shown with three bone screws 55 with the spacer 10 disposed between two adjacent bone screws 55 and the end portions 16 and 18 each within the U-shaped channels of the three bone screws 55. A closure structure 57 is then inserted into and advanced between the arms of each of the bone screws 55. The closure structure 57 is rotated, using a tool (not shown) engaged with the inner drive until a selected pressure is reached at which point the portion 16 or 18 is urged toward, but not completely seated in the U-shaped channels of the bone screws 55. For example, about 80 to about 120 inch pounds pressure may be required for fixing the bone screw shank 60 with respect to the receiver 61 at a desired angle of articulation.

The assembly 1 is thus substantially dynamically loaded and oriented relative to the cooperating vertebra, providing relief (e.g., shock absorption) and protected movement with respect to distraction, compressive, torsion and shear forces placed on the assembly 1 and the connected bone screws 55. The spacer 10 and cooperating core 8 and fins 24 and 26 allows the assembly 1 to twist or turn, providing some relief for torsional stresses. The spacer 10 in cooperation with the fins 24 and 26, however limits such torsional movement as well as compression and distraction displacements, providing spinal support. The core 8 further provides protection against sheer stresses placed on the assembly 1.

If removal of the assembly 1 from any of the bone screw assemblies 55 is necessary, or if it is desired to release the assembly 1 at a particular location, disassembly is accomplished by using the driving tool (not shown) with a driving formation cooperating with the closure structure 57 internal drive to rotate and remove the closure structure 57 from the receiver 61. Disassembly is then accomplished in reverse order to the procedure described previously herein for assembly.

Eventually, if the spine requires even more stiff support, the connecting member assembly 1 according to the invention may be removed and replaced with another longitudinal connecting member, such as a stiff, solid integral rod, having the same diameter as the end portions 16 and 18, utilizing the same receivers 61 and the same or similar closure structures 57. Alternatively, if less support is eventually required, a less rigid rod having the same diameter as the portions 16 and 18, may replace the assembly 1, also utilizing the same bone screws 55.

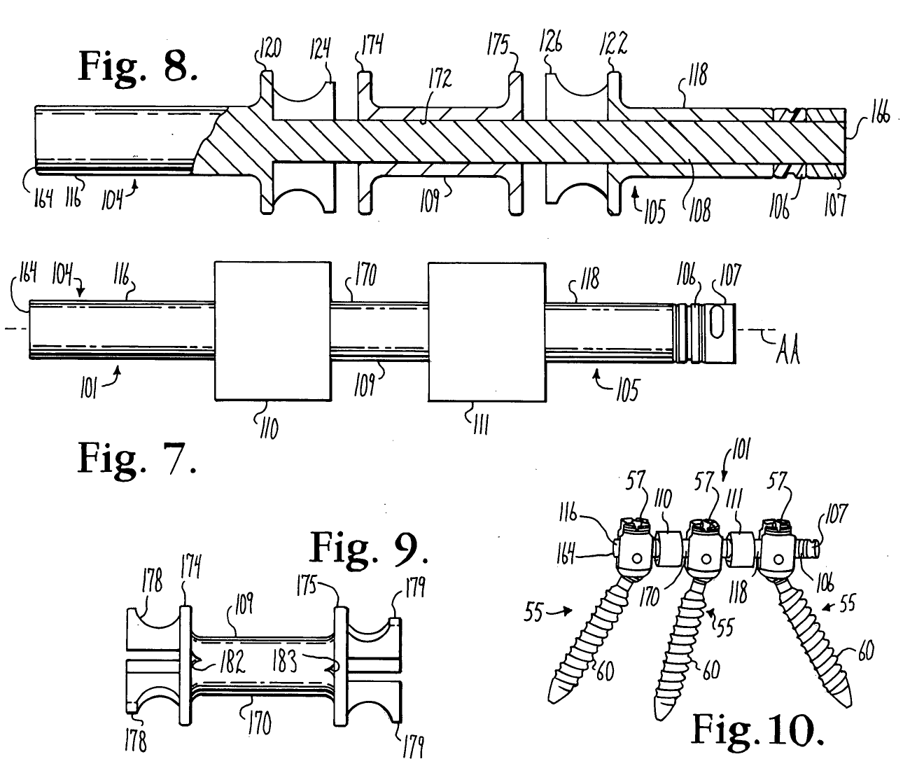

With reference to FIGS. 7-10, the reference numeral 101 generally designates a second embodiment of a non-fusion dynamic stabilization longitudinal connecting member assembly according to the present invention. The connecting member assembly 101 includes first and second elongate segments, generally 104 and 105, an elastic bumper 106, a crimping ring 107, and a solid inner core extension 108, identical or substantially similar to respective segments 4 and 5, elastic bumper 6, crimping ring 7 and inner core extension 8 of the assembly 1 previously described herein. The assembly 101 further includes an outer sleeve or tube trolley 109 that is operatively disposed between the segments 104 and 105. As will be described in greater detail below, the sleeve 109 includes fins on either side thereof that cooperate with the fins of the segments 104 and 105, allowing for a longitudinal connector having more than one dynamic portion, each connected by an over-molded spacer. In the embodiment 101, the fins of the segment 104 and one side of the sleeve 109 are surrounded by the over-molded portion 110 and the fins of the segment 105 and the opposite side of the sleeve 109 are surrounded by the over-molded portion 111. The over-molded portions or spacers 110 and ill are each identical or substantially similar in form and function to the spacer 10 previously described herein with respect to the assembly 1.

The illustrated core 108 is substantially cylindrical and substantially stiff and solid, having a central longitudinal axis AA that is also the central longitudinal axis AA of the entire assembly 101 when the spacers 110 and 111 are molded thereon, connecting the segment 104 with the sleeve 109 and the segment 105 with the sleeve 109, with the core slidingly received by and extending through the sleeve 109 and the segment and 105. The core 108 may be tensioned prior to molding of the spacers 110 and 111.

With particular reference to FIG. 8, similar to the segments 4 and 5, the elongate segments 104 and 105 further include respective bone attachment end portions 116 and 118, respective end plates 120 and 122 having respective integral hooked fin or wing members 124 and 126. In the illustrated embodiment, there are three equally spaced fins 124 and 126 extending generally along the axis AA from the respective plates 120 and 122. However, in other embodiments according to the invention there may be more than three or less than three hooked fins 124 and 126. The segment 104 further includes an end 164 that is opposite an end 166 of the core 108. The illustrated central core 108 is integral with the plate 120 and extends along the central axis AA and between both sets of fins 124 and 126 and through the sleeve 109.

With particular reference to FIGS. 8 and 9, the stiff sleeve or tube trolley 109 includes a substantially cylindrical body 170 having an inner lumen or through bore 172 that operatively extends along the axis AA. The sleeve 109 includes a first end plate 174 and an opposite end plate 175. The end plates 174 and 175 have respective integral hooked fin or wing members 178 and 179. In the illustrated embodiment, there are three equally spaced fins 178 and 179 extending generally along the axis AA from the respective plates 174 and 175 that are substantially similar in size and shape with the hooked fins 124 and 126 and the fins 24 and 26 of the assembly 1. However, in other embodiments according to the invention there may be more than three or less than three hooked fins 178 and 179. In operation, the illustrated central core 108 extends along the central axis AA between both sets of fins 178 and 179 and is slidingly received in the through bore 172. Each plate 174 and 175 also includes three elastomer receiving apertures or through bores 182 and 183, respectively, spaced substantially equally between the respective fins 178 and 179. The through bores 182 and 183 extend substantially parallel to the axis AA.

With reference to FIG. 10, in use, at least three bone screws 55 are implanted into vertebrae for use with the longitudinal connecting member assembly 101 in the same or similar manner as previously discussed herein with respect to the assembly 1. With reference to FIG. 8, the longitudinal connecting member assembly 101 may be assembled to provide a neutral core 8 and neutral spacers 110 and 111 or a pre-tensioned core 108 and pre-compressed spacers 110 and 111 and bumper 106 prior to implanting the assembly 101 in a patient. Pre-tensioning is accomplished by first providing the segment 104 with a core that is longer in the axial direction AA than the core 108 illustrated in the drawing figures so that the core 108 may be gripped during compression of the spacers 110, 111 or bumper 106 and crimping of the ring 107 onto the core 108. In all installations, the assembly 101 is assembled by threading the sleeve 109 onto the core 108, followed by threading the segment 105 onto the core 108 with the fins 124 of the segment 104 facing the fins 178 of the sleeve 109 and the fins 126 of the segment 105 facing the fins 179 of the sleeve 109. The core 108 is slidingly received in the bores of the sleeve 109 and the segment 105. The facing fins are manipulated to be evenly spaced from one another with a desired uniform space between the fin ends and facing plates. This is performed in a factory setting with the end portions 116 and 118 and sleeve body 170 held in a jig or other holding mechanism that frictionally engages and holds the sections 116 and 118 and the sleeve 109, for example, and the spacer 110 is molded about the plates 120 and 174 as well as the fins 124 and 178 and the spacer 111 is molded about the plates 122 and 175 as well as the fins 126 and 179. The elastomer of the spacers 110 and 11 flows through the bores formed in the plates as well as around and about each of the fins 124, 126, 178 and 179, the resulting molded spacers 110 and 111 surrounding all of the fins surfaces and at least partially and up to fully surrounding the surfaces of the plates 120, 122, 174 and 175. If desired, prior to molding, a sheath or coating may be placed about the core 108 so that the elastomeric material of the spacers 110 and 111 does not contact the core 108. However, in other embodiments of the invention, the elastomer is allowed to flow about and contact the core 108, that may be pre-tensioned or tensioned after the molding process. The jig or holding mechanism may then be released from the portions 116 and 118 and the sleeve body 170 after the molding of the spacers 110 and 111 is completed. The portions 116 and 118 and the body 170 of the sleeve 109 may be held in straight (axial along AA) or angled positions with respect to one another.

Either before or after molding, the bumper 106 is loaded onto the core 108 and moved along the core 108 until the bumper 106 contacts the end portion 118. The crimping ring 107 is thereafter loaded onto the core 108 until the ring 107 abuts against the bumper 106. Manipulation tools (not shown) are then used to grasp the core 108 near the end 166 and at the bone anchor attachment portion 116, placing tension on the core 108, if desired. Furthermore, the spacers 110 and 111 and/or the bumper 106 may be compressed, followed by deforming the crimping ring at the crimp grooves thereof against the core 108 as previously described herein with respect to the crimp ring 7 and core 8 of the assembly 1.

With reference to FIG. 10, the assembly 101 is eventually positioned in an open or percutaneous manner in cooperation with three bone screws 55 with the spacer 110 disposed between two adjacent bone screws 55 and the spacer 111 disposed between two adjacent bone screws 55 with the end portions 116 and 118, and the sleeve body 170 each within the U-shaped channels of one of the three bone screws 55. A closure structure 57 is then inserted into and advanced between the arms of each of the bone screws 55. The closure structure 57 is rotated, using a tool (not shown) engaged with the inner drive until a selected pressure is reached at which point the portion 16 or 18 is urged toward, but not completely seated in the U-shaped channels of the bone screws 55. For example, about 80 to about 120 inch pounds pressure may be required for fixing the bone screw shank 60 with respect to the receiver 61 at a desired angle of articulation.

The assembly 101 is thus substantially dynamically loaded and oriented relative to the cooperating vertebra, providing relief (e.g., shock absorption) and protected movement with respect to distraction, compressive, torsion and shear forces placed on the assembly 101 and the connected bone screws 55. The spacers 110 and 111 and cooperating core 108 and fins (124 and 178; and 126 and 179) allow the assembly 101 to twist or turn, providing some relief for torsional stresses. The spacers 110 and 111 and cooperating over-molded fins, however limit such torsional movement as well as compression and distraction, providing spinal support. The solid stiff core 108 further provides protection against sheer stresses placed on the assembly 101.

If removal of the assembly 101 from any of the bone screw assemblies 55 is necessary, or if it is desired to release the assembly 101 at a particular location, disassembly is accomplished by using the driving tool (not shown) with a driving formation cooperating with the closure structure 57 internal drive to rotate and remove the closure structure 57 from the receiver 61. Disassembly is then accomplished in reverse order to the procedure described previously herein for assembly.

It is to be understood that while certain forms of the present invention have been illustrated and described herein, it is not to be limited to the specific forms or arrangement of parts described and shown.

What is claimed and desired to be secured by Letters Patent is as follows:

1. In a medical implant assembly having at least two bone attachment structures cooperating with a longitudinal connecting member, the improvement wherein the longitudinal connecting member comprises:
a) first and second segments, each segment having at least one fin extending axially therefrom, the second segment having a through bore;
b) an inner core extension fixed to the first segment and extending through the through bore of the second segment; and
c) a molded elastomer substantially surrounding each fin and a portion of the inner core extension.

2. The improvement of claim 1 wherein the at least one fin is a plurality of fins.

3. The improvement of claim 1 further comprising an elastic bumper receiving the inner core at an end thereof.

4. The improvement of claim 3 wherein at least one of the the elastic bumper and the molded elastomer is held in a compressed state by a structure fixed to the inner core extension.

5. The improvement of claim 4 wherein the structure fixed to the inner core extension is a crimping ring.

6. The improvement of claim 1 wherein the inner core extension is integral with the first segment.

7. The improvement of claim 1 further comprising a sleeve disposed between the first and second segments, the sleeve having a lumen, the inner core slidingly receivable in the lumen, the molded elastomer being in first and second portions, the first portion attaching the first segment to the sleeve and the second portion attaching the second segment to the sleeve.

8. The improvement of claim 7 wherein the sleeve has at least one fin extending axially therefrom.

9. The improvement of claim 7 wherein the sleeve has at least two fins extending from opposite ends of the sleeve.

10. In a medical implant assembly having at least two bone attachment structures cooperating with a longitudinal connecting member, the improvement wherein the longitudinal connecting member comprises:
a) first and second elongate segments, the segments aligned along a central axis, each segment having at least one fin extending axially therefrom and radially from the axis, the fins in spaced, overlapping relation along the axis;
b) a molded elastomer substantially surrounding each fin; and
c) an inner core extension fixed to the first segment and extending through the second segment along the central axis.

11. The improvement of claim 10 wherein the at least one fin is a plurality of fins.

12. The improvement of claim 10 wherein the at least one fin is at least a pair of fins on each elongate segment, the fins of the first segment disposed between the fins of the second segment.

13. The improvement of claim 12 wherein the fins are in substantially equal spaced relation to one another.

14. The improvement of claim 10 wherein the at least one fin has a concave surface.

15. The improvement of claim 14 wherein the concave surface faces outwardly away from the axis.

16. The improvement of claim 10 wherein each elongate segment has at least one end plate and the at least one fin extends axially from the end plate.

17. The improvement of claim 16 wherein the molded elastomer surrounds each end plate.

18. In a medical implant assembly having at least three bone anchors cooperating with a longitudinal connecting member, the improvement wherein the longitudinal connecting member comprises:
a) a first elongate member having a first axis, the member sized and shaped for attachment to at least one bone anchor, the elongate member having a first end plate and a first curvate fin fixed to the end plate, the curvate fin extending along the first axis and radially outward from the first axis;
b) a second elongate member having a second axis, the second member sized and shaped for attachment to at least one bone anchor, the second elongate member having a second end plate and a second curvate fin fixed to the second end plate, the second curvate fin extending along the second axis and radially outward from the second axis;
c) a sleeve sized and shaped for attachment to at least one bone anchor, the sleeve disposed between the first and second elongate members, the sleeve having third and fourth end plates, a third curvate fin extending from the third plate and a fourth curvate fin extending from the fourth plate;
d) a first molded elastomeric spacer surrounding the first and third curvate fins and holding the first and third fins in substantially spaced relation with one another;
e) a second molded elastomeric spacer surrounding the second and fourth curvate fins and holding the second and fourth fins in substantially spaced relation to one another; and
f) an inner core extension integral with the first elongate member and slidingly received in the sleeve and the second elongate member.

19. The improvement of claim 18 further comprising
a) an elastic bumper slidingly received on the inner core extension near an end thereof; and
b) a crimping structure abutting the bumper and fixed to the inner core extension.

20. The improvement of claim 18 wherein the first molded elastomer surrounds at least a portion of the first end plate and the third end plate.

21. The improvement of claim 18 wherein the second molded elastomer surrounds at least a portion of the second end plate and the fourth end plate.

22. The improvement of claim 18 wherein the first fin is a plurality of fins and the third fin is a plurality of fins, each first fin being at least partially disposed between a pair of third fins.

23. The improvement of claim 18 where in the second fin is a plurality of fins and the fourth fin is a plurality of fins, each second fin being at least partially disposed between a pair of fourth fins.
